(19) United States
(12) Patent Application Publication
(10) Pub. No.: US 2015/0219553 A1
(43) Pub. Date: Aug. 6, 2015

(54) Laser Based, Temperature Insensitive, Carbon Dioxide Isotope Ratio Measurement

(71) Applicant: Southwest Science Incorporated, Santa Fe, NM (US)

(72) Inventors: Steven Michael Massick, Placitas, NM (US); Kristen A. Peterson, Santa Fe, NM (US); Anthony L. Gomez, Santa Fe, NM (US); Joel A. Silver, Santa Fe, NM (US)

(21) Appl. No.: 14/686,297

(22) Filed: Apr. 14, 2015

Related U.S. Application Data
(63) Continuation of application No. 13/482,655, filed on May 29, 2012.
(60) Provisional application No. 61/490,348, filed on May 26, 2011.

(51) Int. Cl. G01N 21/3504; G01N 33/497; G01N 21/39

(52) U.S. Cl. CPC G01N 21/3504; G01N 21/39; G01N 33/497; G01N 2021/399; G01N 2201/0612

(57) ABSTRACT

An apparatus and method (and related kit) for determination of the isotopic ratio of ¹³C to ¹²C in a gas sample containing carbon dioxide comprising introducing gas into a gas sample chamber, directing light into the sample chamber from a laser light source, the laser light source being capable of accessing one or more of the wavelength pairs 2054.37 and 2052.42; 2054.96 and 2051.67; or 2760.53 and 2760.08 nanometers, and with a detector detecting the laser light energy after passage through the sample chamber.

### CROSS-REFERENCE TO RELATED APPLICATIONS

This application is a continuation of U.S. patent application Ser. No. 13/482,655, entitled “Laser Based, Temperature Insensitive Carbon Dioxide Isotope Ratio Measurement”, filed May 29, 2012, which claims priority to and the benefit of the filing of U.S. Provisional Patent Application Ser. No. 61/490,348, entitled “Laser Based, Temperature Insensitive, Carbon Dioxide Isotope Ratio Measurement”, filed on May 26, 2011, and the specifications and claims thereof are incorporated herein by reference.

### STATEMENT REGARDING FEDERALLY SPONSORED RESEARCH OR DEVELOPMENT

This invention was made with government support under Contract Nos. NNX12CE29P and NNX11CD28P awarded by the National Aeronautics and Space Administration. The government has certain rights in the invention.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The present invention relates to methods and apparatuses for measuring carbon dioxide isotope ratios.

2. Description of Related Art

The present invention is directed to devices and systems for the precise measurement of ¹³C/¹²C isotopic ratios of gaseous carbon dioxide samples (δ¹³CO₂) Determination of such ratios, typically expressed as per thousand Too, are of great importance to many fields such as, but not limited to, geology, medicine, paleoclimatology, and atmospheric science. Carbon dioxide is recognized as an anthropogenic greenhouse gas and analysis of δ¹³CO₂ is appropriate for enforcing constraints on the global CO₂ budget. In addition, geologists have recognized that carbon dioxide emanating from volcanic activity is depleted in ¹³CO₂. Thereby, volcanic activity can be monitored and forecast by analyzing both the amount of CO₂ and the δ¹³CO₂ of gases emanating from the soil in volcanic craters. Further, an accepted non-invasive medical diagnostic for human gastrointestinal H. Pylori infections uses an increase in the δ¹³CO₂ of exhaled breath following ingestion of ¹³C-labeled urea as indication of infection. Bell, G. D., et al., “14C-urea breath analysis, a non-invasive test for Campylobacter pylori in the stomach”, Lancet, 1987, 1: p. 1367-1368. See, also, U.S. Pat. No. 5,929,442, to Higashi; and U.S. Pat. No. 6,800,855, to Dong et al.

The present devices and systems permit the use of advanced methods for the determination of the isotopic ratios such that some or all of improved accuracy, convenience, portability, energy consumption, and applicability may be enjoyed.

Field deployable instrumentation for environmental, atmospheric research that precisely measures δ¹³CO₂ can be used to monitor the location, magnitude, and origin of carbon sources and sinks. The characterization of carbon sinks as oceanic or terrestrial is possible since photosynthesis discriminates against ¹³C and, relative to the atmosphere, plants have a lower isotopic ratio than the atmosphere. Thus, uptake of CO₂ by plants results in higher atmospheric δ¹³CO₂. Oceanic uptake, however, shows little discrimination between the carbon isotopes. By examining variability in isotope measurements it is possible to identify the magnitude and type of carbon sink. Isotopes of CO₂ are now routinely measured in global National Oceanic and Atmospheric Administration (NOAA) sampling campaigns and have offered significant insight into regional and global sources and sinks. However, little information exists on CO₂ isotopes on smaller geographic scales and shorter timeframes.

The best known means for CO₂ isotope measurements is isotope ratio mass spectrometry (IRMS). However, the instrumentation associated with IRMS is expensive, heavy, and requires a skilled technician to operate. IRMS is typically confined to laboratory settings. The complexity of IRMS requires on-site sample collection followed by laboratory analysis of samples at a location typically distant from the collection site. This has limited the application of δ¹³CO₂ measurements as a widely deployable research tool.

Optical absorption spectroscopic methods have been used to determine the δ¹³CO₂ of gas samples. Single isotope measurement is possible because the light absorbed by ¹³CO₂ is slightly shifted in wavelength from that of ¹²CO₂. The optical methods of δ¹³CO₂ determination vary from nondispersive measurements of light absorption of entire rotational-vibrational bands of ¹³CO₂ and ¹²CO₂ using broadband light sources, to diode laser based measurements of a single absorption line of both ¹³CO₂ and ¹²CO₂. These measurements are typically done in the near or mid-infrared wavelength regions. The diode laser based δ¹³CO₂ measurement method offers development of widely deployable, low power δ¹³CO₂ instruments. However, a significant limitation to the extant diode laser based methods is that the δ¹³CO₂ values determined can be highly temperature dependent, at times requiring gas temperatures to be regulated to within 0.01° K. This temperature sensitivity limits the accuracy of the δ¹³CO₂ measurement. Apparatuses and systems which can operate under less limited temperature restrictions, yet offering improvements in size, weight, power consumption and cost, are very much desired. It is greatly desired to have improved diode-laser-based δ¹³CO₂ instruments, systems and methods which provide some or all of the foregoing, desired improvements.

### BRIEF SUMMARY OF THE INVENTION

The present invention is of an apparatus and method for determination of the isotopic ratio of ¹³C to ¹²C in a gas sample containing carbon dioxide, comprising: introducing gas into a gas sample chamber; directing light into the sample chamber from a laser light source, the laser light source being capable of accessing one or more of the wavelength pairs 2054.37 and 2052.42; 2054.96 and 2051.67; and 2760.53 and 2760.08 nanometers; and with a detector detecting the laser light energy after passage through the sample chamber. In the preferred embodiment, a processor interprets or presents the signals received by the detector. One or more of the following are employed: power supply, gas pump, pressure gauge, signal processor, and reference gas chamber. The laser light source scans the pair of wavelengths using wavelength modulation spectroscopy. The laser light source preferably comprises a single laser emitter and is a vertical cavity surface emitting laser. Alternatively, the laser light source comprises a pair of laser emitters. The invention is preferably controlled with a digital computer.

The present invention is also of a kit comprising an apparatus for determination of the isotopic ratio of ¹³C to ¹²C in a gas sample containing carbon dioxide comprising a gas sample chamber, a laser light source and a detector for laser light energy, the laser light source being capable of scanning one of the wavelength pairs 2054.37 and 2052.42; 2054.96 and 2051.67 or 2760.53 and 2760.08 nanometers and a plurality of gas collection containers or devices.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 is a plan view of an exemplary laser absorbance device in accordance with some embodiments of this invention; and

FIG. 2 illustrates a preferred jump scanning regime.

### DETAILED DESCRIPTION OF THE INVENTION

The present invention utilizes small, low power, near infrared diode lasers to attain field portable, battery operated δ¹³CO₂ measurement instruments with high degrees of accuracy and sensitivity. These devices and the methodologies which employ them may be used to determine δ¹³CO₂ in diverse environments and for diverse useful purposes. Carbon isotope gas measurement devices are now provided that are on the order of one quarter of the size and weight of the commercial POCone device available from Meretek Diagnostics Inc. for measuring breath carbon dioxide isotope ratios, and one quarter the size and one tenth the weight of carbon dioxide isotope analyzers available commercially from Los Gatos Research, Inc. Further the present devices can use far less power than the existing commercial devices. The present δ¹³CO₂ devices have sensitivities of from about 0.2 to 0.3 ‰ a figure appropriate for monitoring gases in industrial, environmental, medical and other milieus.

The present invention provides laser-based, optical absorption methods of analyzing carbon isotope ratios in carbon dioxide samples that are not adversely affected by temperature changes. The accuracy and precision of measuring carbon dioxide isotope ratios can be affected by changes in the ground state population of carbon dioxide. The origins of the isotopic differences in samples may be diverse and are not the subject of the present invention. Rather, it is recognized that ascertaining the value of the isotopic ratio is inherently important and commercially useful. The present invention provides greatly improved devices and methods for accomplishing this goal irrespective of the sources of gas samples or the evaluative objective to be attained.

The relatively small size, light weight, temperature insensitivity, low power consumption and other features of detection instruments in accordance with one or more embodiments of this invention lend to their desirability. The present devices provide the opportunity to perform δ¹³CO₂ measurements in new ways and to employ such measurements to attain knowledge about diverse samples quickly, inexpensively, accurately and in a manner which benefits from the deployability of the devices. It is to be understood, however, that the invention may be practiced in different ways and that not all benefits may be enjoyed by all such embodiments.

Optical absorption spectroscopy is based on the well-known Beer-Lambert Law. Gas concentrations are determined by measuring the change in the laser beam intensity, I₀, due to optical absorption of the beam by a sample of the gas. If a sample cell is used for the analysis, such that the path length of the beam and inherent characteristics of the measuring device are constant, absorbance measurements allow calculation of the gas number density, n, or gas concentration.

Diode laser-based gas-phase absorption measurements interrogate individual absorption lines of gas molecules. These absorption lines correspond to the transition of the gas molecule, e.g. carbon dioxide, from a ground energy state to a higher excited energy state by absorption of a photon of light. The lines are typically quite narrow at reduced sample gas pressure thereby permitting selective detection of a gas in the presence of other background gases such as water vapor. The isotopes of CO₂ have distinct absorption lines that occur at shifted wavelengths with respect to each other due to the mass difference between ¹²C and ¹³C.

It is now appreciated to be of great importance that absorbance measurements are affected by the gas temperature and that the magnitude of this temperature sensitivity varies depending on absorption line selection and the total ground state energy of the optical transition. A collection of molecules at room temperature is distributed over many discrete molecular energy states that vary in total energy according to how fast the molecules rotate and vibrate. That is, the ground state molecular population is distributed about discrete rotational and vibrational energy states according to a Boltzmann distribution.

It is now appreciated that a significant temperature dependence of δ¹³CO₂ can seriously affect the long term stability and sensitivity of diode laser-based isotopic measurements of carbon dioxide. Chleboun, J. and P. Kocna, “Isotope selective nondispersive infrared spectrometry can compete with isotope ratio mass spectrometry in cumulative ¹³CO₂ breath tests: assessment of accuracy”, Kin. Biochem. Metab., 2005, 13(34): p. 92-97; Castrillo, A., et al., “Measuring the ¹³C/¹²C isotope ratio in atmospheric CO₂ by means of laser absorption spectrometry: a new perspective based on a 2.05-μm diode laser”, Isotopes in Environmental and Health Studies, 2006, 42(1): p. 47-56; Gagliardi, G., et al., “High-precision determination of the ¹³CO₂/¹²CO₂ isotope ratio using a portable 2.008-μm diode-laser spectrometer”, Appl. Phys. B, 2003, 77: p. 119-124; Horner, G., et al., “Isotope selective analysis of CO2 with tunable diode laser (TDL) spectroscopy in the NIR” Analyst, 2004, 129: p. 772-778; and Wahl, E. H., et al., “Applications of cavity ring-down spectroscopy to high precision isotope ratio measurement of ¹³C/¹²C in carbon dioxide”, Isotopes in Environmental and Health Studies, 2006, 42: p. 21-35. It is to be noted that Castrillo et al. achieved δ¹³CO₂ measurements with a short term precision of 0.3‰ considering that the CO₂ absorption lines they selected in the 2 micrometer band have a 280 wavenumber (cm⁻¹) ground state energy difference that results in a temperature sensitivity of 4.6‰ per degree Kelvin. This temperature dependence resulted in a long term δ¹³CO₂ reproducibility of 1‰. However, gas temperature can be precisely controlled in laboratory settings but such is not amenable to portable, low power instrumentation.

The present inventors have determined that ¹³CO₂ and ¹²CO₂ absorption lines with near equal ground state energies can be useful in attaining relative temperature insensitivity for isotopic ratio measurements. By doing this, the sensitivity limitations imposed by the absorption cross section temperature dependence have been largely avoided. However, diode lasers have a limited current tuning scan range, especially for distributed feedback diode lasers that have small current tuning ranges of 1 to 2 cm⁻¹ used in the δ¹³CO₂ measurement studies noted above.

Vertical cavity surface emitting lasers (VCSELs) have been shown to attain scan ranges of 10 to 15 cm⁻¹. These have been used to give rise to rugged, high precision field instruments as exemplified by a laser hygrometer manufactured by Southwest Sciences, Inc. flown on a National Science Foundation airplane and a field-deployable methane analyzer manufactured by LI-COR. Accordingly, for certain of the preferred embodiments of the present invention, VCSELs have been fabricated which may be scanned over the desired spectral wavelengths, at a useful scan rate in the context of an overall testing apparatus as to give rise to some or all of the desired benefits of the present invention. In some embodiments, the VCSEL devices are caused to scan in the kilohertz scan rate or greater over approximately 10 cm⁻¹ ranges.

Suitable laser sources may also be formed from a plurality, usually a pair, of laser emitters. Such emitters may be fabricated to emit at one of the preferred wavelengths of a wavelength pair. VCSEL devices useful in the invention may be ordered from Vertilas GmbH of Germany and can also be made by other sources of laser emitters.

The present inventors have identified pairs of ¹³CO₂ and ¹²CO₂ spectral lines, each pair of which has near zero ground state energy difference, a line separation less than 12 cm⁻¹, and is substantially free of water interference. It is now been discovered that these pairs of lines are highly useful in the ascertainment of ¹³CO₂/¹²CO₂ isotopic ratios in gas samples. The temperature dependence of measurement using these pairs is desirably low.

It has now been determined that spectral line pairs as follows are highly useful in making carbon dioxide isotopic absorption measurements using diode lasers in gas cells in accordance with embodiments of this invention:

| ¹²CO₂ wavelength (nm) | ¹³CO₂ wavelength (nm) |
|---|---|
| 2054.37 | 2052.42 |
| 2054.96 | 2051.67 |
| 2760.53 | 2760.08 |

It will be appreciated that the wavelengths identified in the foregoing line pairs are nominal and that some variation from the listed values may be useful. In this regard, it will be understood that useful wavelengths will be those which are sufficiently close to the recited values as to provide one or more of the benefits of the present invention. Thus, such wavelengths will confer either improved accuracy, improved temperature stability or another of the desirable properties set forth herein to the measurement of CO₂ isotopic ratios. In general, preferred wavelengths will be within 0.5 of a nanometer of the recited values.

In addition to the laser light source operating at the desired wavelengths, the present devices preferably include a sample container for holding the gas sample, which container is configured to provide a relatively long light path through the sample by way of mirrors. One or more signal detectors are included as is control circuitry for controlling the laser and for collecting and manipulating the output signal from the detector or detectors. Other equipment to facilitate sample collection, sample preparation, data interpretation and display and other things may also be included in systems and kits provided by this invention. All such components are preferably sufficiently rugged as to permit the deployment of the devices outside of a laboratory and even in a hand held context.

The present apparatuses are also useful in a system or kit. Components of the system or kit may include sample collection containers, such as gas tight bags, preferably ones featuring injection ports, syringes, and other items which facilitate sample collection and transfer to the sample chamber of the apparatus. Such sample collection elements may assume different configurations depending upon the source of the gas to be sampled. Thus, the same may, for example, be useful for collecting breath of a patient.

Portable devices and systems are known having a general arrangement of elements suitable for use in some of the embodiments of the present invention. For example, the '96 Hawk hand-held methane leak detector system sold by Southern Cross Corp. provides sample container, mirror assemblies, power supply, sample handling and other components which may be adapted for use in the invention. Such systems, however, are not otherwise amenable for such use. Thus, the provision of diode laser sources which are capable of scanning the requisite spectral line pairs with effective frequency, stability and accuracy must be accomplished. Likewise, detectors for sensing optical absorption in the selected line pairs with needed accuracy as well as data collection, storage, manipulation and display or reporting devices and/or software is needed.

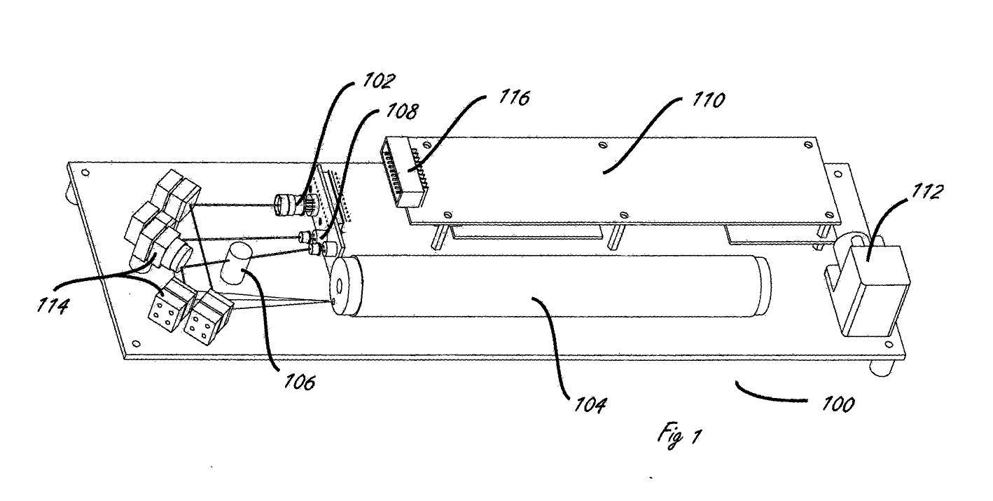

FIG. 1 depicts certain aspects of one device in accordance with this invention. A CO₂ optical absorption measurement device is depicted 100, which comprises a diode laser source 102, mirrors 114, and gas sample chamber 104. Taken together, these form an optical path in conjunction with preferred reflective surfaces inside the sample chamber, not shown. The optical path, which is effectively many times longer than the physical length of the chamber, permits the enhanced absorption of laser light by gas samples in the chamber. One or more gas pumps, 112 are conveniently included to transport gas sample into and out of the sample chamber which may, likewise, be provided with one or more pressure gauges. Preferably, a reference gas chamber, 106 is also employed together with mirrors, 114 for directing laser light through the reference gas chamber 106. The light paths through the sample and reference chambers are directed to one or more detectors, 108 for assessing the intensity of laser light. Processor or processors in control module, 110 determine the amount of absorption of incident laser light by the sample in the sample chamber, by reference to the reference sample in the reference chamber. This determination may be performed by routine software of firmware, either on board the device or external to it. Preferably, electrical connections, 116 are provided enabling either signals or processed data from the device to be ported to external display or data collection and manipulation devices. In accordance with certain preferred embodiments, some or all of the elements making up apparatuses and systems of the invention and the functions they perform are operated under the control of a controller. Such controller, which may be on board the instrument or external to it, may be a general purpose digital computational device or a special purpose digital or digital-analog device or devices. Control by the controller may be of, for example, power supplies for the laser, detector, gas sample pump, processors and other components.

In operation, a gas sample suspected of containing carbon dioxide is introduced into the sample chamber of the devices of the invention. The gas may be held in the sample chamber for a period of time or flow continuously. The laser light source or sources is then caused to transit the sample chamber, preferably via a multiply reflecting pathway so as to increase the overall path length and improve the measurement sensitivity. The light source is then directed to one or more sensors and the sensor readings interpreted to give rise to a value for wavelength absorption by the sample. The methodologies for making this determination are well known in the art, and include, for example, direct absorption spectroscopy, wavelength modulation spectroscopy, cavity ringdown spectroscopy, and other alternatives. By comparing the absorption of light having each of the chosen pair of wavelengths, values for the carbon 12 and carbon 13 isotopes in the carbon dioxide sample become known. Perforce, their ratio may be calculated. For some of the preferred embodiments of the invention, a reference gas sample is provided and the same irradiated, detected and the signal interpreted. The data thus obtained is used to standardize the data arising from irradiation of the sample chamber.

The mechanics of the apparatus including the supply of power to the laser light source or sources, to the detectors and to any data storage, presentation and manipulation elements is preferably under the control of a controller, whether digital or analog. A digital computer may also or in addition be used. Such computer may be on board or connected via a control interface.

It is preferred that determination of light absorption in accordance with the present invention be accomplished by wavelength modulation spectroscopy (WMS). While WMS has been used previously for δ¹³CO₂ measurements, it has never been performed for the line pairs that have now been determined to be used for isotopic ratios determinations in carbon dioxide.

WMS is preferred to direct absorption spectroscopy for use in the present invention, although direct measurement may be used if desired. For direct absorbance measurements the laser current is ramped so that the wavelength output is repeatedly scanned across a gas absorption line and the spectra generated are co-averaged. Analysis of direct absorption spectra involves detecting small changes on a large detector signal. For very low concentration changes this is problematic. To perform WMS, a small high-frequency modulation is superimposed on the diode laser current ramp. This current modulation produces a modulation of the laser wavelength at the same high frequency. Absorption by the target gas converts the wavelength modulation to an amplitude modulation of the laser intensity incident on the detector, adding AC components to the detector photocurrent. The detector photocurrent is demodulated at twice the modulation frequency, 2f detection. This selectively amplifies only the AC components (a zero background measurement) and shifts the measurement from near DC to higher frequencies where laser noise is reduced. Spectral noise is greatly reduced by performing signal detection at frequencies (>10 kHz) high enough to avoid fluctuations in the laser output power, laser excess (1/f) noise. In carefully optimized laboratory setups, WMS has measured absorbances as low as 1×10⁻⁷, which is near the detector noise limit. However, in compact field instrumentation, background artifacts typically limit the minimum detectable absorbance αₘᵢₙ to 1×10⁻⁵ s^{1/2}. The value for αₘᵢₙ can be improved by longer time averaging of the 2f signal with the improvement scaling as t^{1/2} for periods of 100 to 300 seconds.

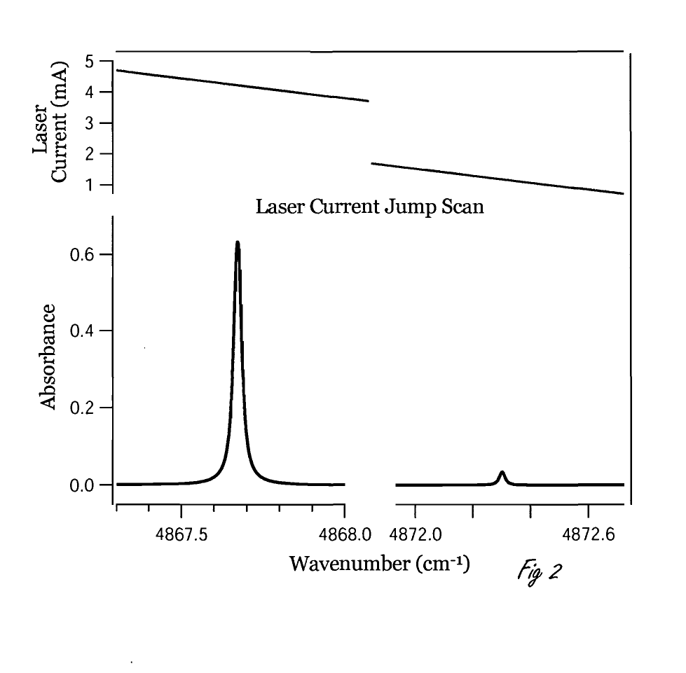

The ¹³CO₂ and ¹²CO₂ absorption line pairs which have now been discovered to give rise to relatively temperature insensitive δ¹³CO₂ isotopic ratio determinations in gas samples are separated by several absorption lines that do not need to be measured. Instead of continuously scanning the laser wavelength between the two peaks of interest in each pair, the electronics is caused to operate the laser in a jump scan fashion. This is illustrated in FIG. 2. The laser current scan is programmed to have a discontinuity that will rapidly change the wavelength. The first few data points after the jump are preferably not used, as the laser wavelength may not be stable immediately after the current jump. VCSELs used in the present invention may be operated in this way even with four current jumps in order to measure five different absorption lines simultaneously with no undue reduction in sensitivity.

In the preferred embodiment, and as readily understood by one of ordinary skill in the art, the apparatus according to the invention will include a general or specific purpose computer or distributed system programmed with computer software implementing the steps described above, which computer software may be in any appropriate computer language, including C++, FORTRAN, BASIC, Java, assembly language, microcode, distributed programming languages, etc. The apparatus may also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements.

Note that in the specification and claims, wavelengths are understood to be within 0.5 of a nanometer of the recited values and “about” or “approximately” means within twenty percent (20%) of the numerical amount cited. All computer software employed to effect the methods of the invention may be embodied on any non-transitory computer-readable medium (including combinations of mediums), including without limitation CD-ROMs, DVD-ROMs, hard drives (local or network storage device), USB keys, other removable drives, ROM, and firmware.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A method for determination of the isotopic ratio of ¹³C to ¹²C in a gas sample containing carbon dioxide, the method comprising the steps of:
introducing the gas sample into a gas sample chamber;
directing laser light into the sample chamber from a laser light source, the laser light being in part absorbed by one or more of the carbon dioxide absorption line pairs designated by the wavelength pairs 2054.37 and 2052.42; 2054.96 and 2051.67; and 2760.53 and 2760.08 nanometers; and
with one or more detectors, detecting the remaining laser light after passage through the sample chamber and determining an amount of absorption by the one or more carbon dioxide absorption line pairs.

2. The method of claim 1 further comprising interpreting or presenting signals generated by the detector upon receipt of the laser light energy from the laser light source after passage through the sample chamber with a processor.

3. The method of claim 1 further comprising employing a reference gas chamber receiving laser light energy.

4. The method of claim 1 wherein wavelength modulation spectroscopy is applied to one or more of the carbon dioxide absorption line pairs.

5. The method of claim 1 wherein said one or more of the carbon dioxide absorption line pairs is designated by 2054.37 and 2052.42 nanometers.

6. The method of claim 1 wherein said one or more of the carbon dioxide absorption line pairs is designated by 2051.67 and 2054.96 nanometers.

7. The method of claim 1 wherein said one or more of the carbon dioxide absorption line pairs is designated by 2760.53 and 2760.08 nanometers.

8. The method of claim 1 wherein the laser light source comprises a pair of laser emitters.

9. The method of claim 1 wherein the laser light source is a vertical cavity surface emitting laser.

10. The method of claim 1 additionally comprising controlling the method with a digital computer.
